(19) United States
(12) Patent Application Publication
(10) Pub. No.: US 2002/0174454 A1
(43) Pub. Date: Nov. 21, 2002

(54) Overexpression of ABI5 in plants to prevent precocious seed germination and to confer resistance to drought and high salt

(75) Inventors: Luis Lopez-Molina, New York City, NY (US); Sebastien Mongrand, New York City, NY (US); Nam-Hai Chua, Scarsdale, NY (US)

Correspondence Address: ROTHWELL, FIGG, ERNST & MANBECK, P.C., 1425 K STREET, N.W., SUITE 800, WASHINGTON, DC, 20005, US

(73) Assignee: The Rockefeller University, New York, NY (US)

(21) Appl. No.: 09/813,352

(22) Filed: Mar. 21, 2001

(51) Int. Cl. C12N 15/82; A01H 5/00; C12N 15/29

(52) U.S. Cl. 800/290; 800/278; 536/23.6

(57) ABSTRACT

The bZIP transcription factor ABI5 is shown to confer an enhanced response to exogenous abscisic acid during germination, seedling establishment and subsequent vegetative growth of plants. ABI5 is necessary, but not sufficient, to maintain germinated embryos in a quiescent state, abscisic acid also being required for maintaining the quiescent state. ABI5 production is enhanced by stress including high salt and drought. This protects plants from drought. Plants which overexpress ABI5 are hypersensitive to abscisic acid and retain water more efficiently than wild-type plants.

### BACKGROUND OF THE INVENTION

Abscisic acid (ABA) is a phytohormone regulating the initiation and maintenance of seed dormancy. It also plays an essential role in a plant's response to stress, particularly water deprivation, notably by regulating stomatal aperture (Himmelbach et al., 1998). The publications and other materials used herein to illuminate the background of the invention or provide additional details respecting the practice, are incorporated by reference, and for convenience are respectively grouped in the appended List of References. So far, ABA-insensitive screens have been widely used to identify molecular genetic components of the ABA signal transduction pathway (Kornneef et al., 1984; Finkelstein, 1994). In these screens, mutagenized Arabidopsis seeds were exposed to ABA concentrations that inhibit germination of wild type (WT) seeds, and putative mutants that were able to germinate were isolated (Kornneef et al., 1984; Finkelstein, 1994). These screens have allowed the identification of several ABI (ABA insensitive) genes (Lopez-Molina and Chua, 2000; Finkelstein and Lynch, 2000; Gosti et al., 1999; Finkelstein et al., 1998; Leung et al., 1994; Meyer et al., 1994; Giraudat et al., 1992) and recent studies have established that ABI1 and ABI3 are key players in vegetative and embryonic ABA responses, respectively (Himmelbach et al., 1998; Parcy and Giraudat, 1997).

Nonetheless, few reports have clarified the physiological role of ABA and mechanisms of action triggered by ABA during germination and early seedling growth. We were led to address these issues by the recent cloning and analysis of ABI5 by two independent groups (Lopez-Molina and Chua, 2000; Finkelstein and Lynch, 2000). The abi5 mutation is recessive and ABI5 encodes a putative transcription factor of the basic leucine zipper (bZIP) family (Lopez-Molina and Chua, 2000; Finkelstein and Lynch, 2000). The bZIP region of ABI5 shows extensive homology to previously characterized plant (bZIP) transcription factors capable of activating reporter genes containing ABA-responsive DNA elements (ABREs) (Choi et al., 2000; Kim et al., 1997; Uno et al., 2000). ABI5 also binds to ABREs in vitro and dry seeds of abi5 show reduced transcript levels of ABA- responsive and ABRE-containing late embryonic genes such as AtEm1 and AtEm6 (Lopez-Molina and Chua, 2000; Finkelstein and Lynch, 2000). Together with the ABA-insensitivity of abi5 mutants, these results show that ABI5 is the first bZIP plant factor found to be required in vivo to signal ABA elicited responses.

### SUMMARY OF THE INVENTION

Results are presented which show that ABI5 expression is involved in tolerance of plants to stress such as drought and high salt. Overexpression of ABI5 inhibits germination of seeds during times of stress. Overexpression of ABI5 results in the plants being hypersensitive to abscisic acid such that they respond to low levels of abscisic acid which have no effect on wild type plants.

One aspect of the invention is seeds, seedlings or plants which are resistant to drought because they overexpress ABI5.

A second aspect of the invention is seeds, seedlings or plants which are resistant to high salt conditions because they overexpress ABI5.

Another aspect of the invention is seeds, seedlings or plants which are hypersensitive to abscisic acid because they overexpress ABI5.

Yet another aspect of the invention are methods of delaying germination of a seed by overproducing ABI5 in the seed.

A further aspect of the invention is a method of inhibiting growth of germinated embryos by overproducing ABI5 in the germinated embryos.

The invention also includes methods of protecting a seed, seedling or plant from drought or from high salt concentrations by overexpressing ABI5 in the seed, seedling or plant.

Another aspect of the invention is a method of making a seed, seedling or plant hypersensitive to abscisic acid by overproducing ABI5 in the seed, seedling or plant.

Yet a further aspect of the invention are methods of creating seeds, seedlings or plants which overproduce ABI5, thereby creating seeds, seedlings or plants which are drought tolerant, tolerant to high salt concentrations, and are hypersensitive to abscisic acid.

The invention is also drawn to a method of protecting somatic embryos from prematurely germinating by overexpressing ABI5 in the embryos.

The invention is also drawn to a method of protecting primed seeds from prematurely germinating by overexpressing ABI5 in the primed seeds.

Finally, a further aspect of the invention is a method of monitoring whether somatic embryos have matured properly or whether seeds have been properly primed, the method comprising measuring ABI5 levels in the somatic embryos or primed seeds.

### BRIEF DESCRIPTION OF THE DRAWINGS

FIGS. 1A-B show Northern blot analyses of transcript accumulation in WT Wassilewskija after breaking dormancy followed by growth for 0-5 days in light in the absence (FIG. 1A) or presence (FIG. 1B) of 5 μM ABA.

FIGS. 1C-D show immunoblot analyses of ABI5 protein in WT Wassilewskija after breaking dormancy followed by growth for 0-5 days in light in the absence (FIG. 1C) or presence (FIG. 1D) of 5 μM ABA.

FIG. 2A shows Western blot analyses indicating that ABA regulates ABI5 accumulation post-transcriptionally.

FIG. 2B shows Western blot analyses indicating that ABA prevents ABI5 degradation.

FIGS. 2C and 2D show Western blot analyses indicating that the ABA inhibition of ABI5 degradation acts via inhibition of the 26S proteasome. FIG. 2C shows results obtained using the anti-protease cocktail and FIG. 2D shows results obtained using the anti-protease cocktail plus 26S proteasome inhibitors.

FIG. 2E is an image of ³²P-labeled HA-ABI5 immunoprecipitated protein from seedlings grown in the presence or absence of ABA and analyzed by SDS-PAGE using Phosphorlmager (BioRad). The isolated protein was either untreated or treated with phosphatase.

FIG. 2F is a Western blot of HA-ABI5 protein from seedlings grown in the presence or absence of ABA wherein the isolated protein was untreated or treated with phosphatase.

FIG. 3A shows Western blot analyses of ABI5 accumulation in seedlings transferred to ABA plates at the indicated times post-stratification. Also shown are photographs of representative seedlings 5 days after treatment. Black bar: 0.5 mm.

FIG. 3B is a Western blot analysis showing rapid degradation of ABI5 in seedlings grown in the absence of ABA.

FIG. 3C shows WT Ws seeds germinated in the presence of ABA (5 μM) 5 days post-stratification. Black bar: 0.5 mm.

FIG. 3D shows WT Ws seeds germinated in the presence of ABA (10 μM) 8 days post-stratification. Black bar: 0.5 mm.

FIG. 3E shows WT Ws seedlings 4 and 30 days post-stratification in the presence or absence of ABA (5 μM). Black bar: 0.5 mm.

FIG. 4A is a Western blot analysis for ABI5 from seedlings grown in the absence of added NaCl.

FIG. 4B is a Western blot analysis for ABI5 from seedlings grown in the presence of 200 mM NaCl.

FIG. 4C is a Western blot analysis for ABI5 from seedlings which were water deprived.

FIG. 5A shows growth of WT Ws and WT/35S::HA-ABI5 seeds in the presence of 0, 0.5 or 5 μM ABA for 5 days.

FIG. 5B shows root growth inhibition for 5 day old seedlings grown for 5 days in the presence of 0, 5 or 10 μM ABA. Dark left bars of each pair represent WT Ws plants; light right bars of each pair represent Ws/35S::HA-ABI5 transgenic lines. Standard deviation is shown by narrow bars at top of each dark or light bar.

FIG. 5C shows water loss (% of control) for Ws/35S::HA-ABI5 (o), WT Ws (+) and abi1 (⋄).

FIG. 5D shows seedlings at 5 days growth from seeds in the presence of 5 μM ABA. WT Ws,, abi5, and three lines of abi5-35S: :ABI5 are shown. The lower portion of the figure shows a Western blot of ABI5 protein in these plants. Each lane contained 5 μg protein.

### DETAILED DESCRIPTION OF THE INVENTION

In the present work, we found that ABA regulates ABI5 accumulation and activity during a limited developmental window. Upon ABA removal, ABI5 is rapidly degraded by a process that may involve the proteasome pathway and this coincides with the initiation of vegetative growth. Transgenic plants overexpressing ABI5 are hypersensitive to ABA, with respect to both germination and vegetative growth. We show that ABA delays rather than blocks germination altogether. This hormone also efficiently prevents vegetative growth by arresting development of mature germinated embryos. Moreover, ABI5 is a rate-limiting component of growth arrest, maintaining Arabidopsis seedlings quiescent for at least a month. The results indicate that this ABA-induced and ABI5-dependent quiescence fulfills a developmental checkpoint during which Arabidopsis plants monitor the water status in the environment.

Seed dormancy is a trait of considerable adaptive significance because it maximizes seedling survival by preventing premature germination under unfavorable conditions. Understanding how seeds break dormancy and initiate growth is also of great agricultural and biotechnological interest. Abscisic acid (ABA) plays primary regulatory roles in the initiation and maintenance of seed dormancy. Here we report that the bZIP transcription factor ABI5 confers an enhanced response to exogenous ABA during germination, and seedling establishment, as well as subsequent vegetative growth. These responses correlate with total ABI5 levels. We show that ABI5 expression defines a narrow developmental window following germination, during which plants monitor the environmental osmotic status before initiating vegetative growth. ABI5 is necessary to maintain germinated embryos in a quiescent state thereby protecting plants from drought. As expected for a key player in ABA-triggered processes, ABI5 protein accumulation, phosphorylation, stability and activity are highly regulated by ABA during germination and early seedling growth.

Although the physiological roles of ABA in the establishment and maintenance of seed dormancy are well characterized, little has been known about the mechanisms by which ABA signals these processes. In the classical ABA-insensitive screen (Kornneef et al., 1984; Finkelstein, 1994), researchers have exploited the inhibitory effect of applied ABA on germination and early growth following stratification. It is reasonable to hypothesize that applying ABA may activate the same signal transduction pathways that promote and maintain dormancy during embryogenesis. However, it is equally reasonable to assume that the effect of ABA is in fact multifaceted when applied to mature embryos. This is suggested by the fact that ABI1 and ABI2 are primarily involved with ABA signaling in vegetative tissues although abi1 and abi2 were recovered in the classical germination screen (Kornneef et al., 1984; Meyer et al., 1994; Leung et al., 1997; Parcy et al., 1994).

The results presented below reveal additional levels of complexity underlying the transition of a dormant seed to a young seedling committed to auxotrophy. Indeed, we have delineated a narrow developmental window following stratification during which ABA regulates endogenous ABI5 protein accumulation. This accumulation is due primarily to an increase in ABI5 transcript abundance upon ABA application, but ABA-mediated protein stabilization also contributes. We used ABA application to reveal that ABI5 serves as a molecular marker for a window period when seeds in their normal environment employ ABA responsiveness as an important adaptive mechanism. This indicates that shortly after stratification, winter annual plants such as Arabidopsis, which have already germinated, are still able to prevent adult growth if external environmental conditions suddenly become potentially fatal. Therefore, one can define at least two early growth checkpoints in Arabidopsis. Passage through the first checkpoint results in germination, as defined by the emergence of the radicle from the seed coat. After germination, a second checkpoint comes into play where water availability in the environment is monitored. Results presented here provide evidence that ABA and ABI5 are key players in the latter process. We have found that the narrow developmental window allowing growth arrest to occur still operates in darkness. This indicates that the window of responsiveness is not a simple consequence of a down regulation of ABA-mediated gene expression by light and strengthens the idea that the growth arrest is a bona fide adaptive response of Arabidopsis.

We present several lines of evidence showing the key role played by ABI5 in the growth arrest. First, in the concentration range (3 to 10 μM) used to isolate ABI mutants (Kornneef et al., 1984; Lopez-Molina and Chua, 2000; Finkelstein and Lynch, 2000), we found that ABA is in fact more efficient as an early growth inhibitor than as an inhibitor of germination. Indeed, we have shown that in the presence of ABA, all seeds eventually fully germinate after 10 days but the germinated embryos remain quiescent for much longer. As soon as ABA is removed, growth and greening are initiated. Second, germinated and ABA-arrested embryos are more tolerant of water stress than control plants not treated with ABA. In nature, winter annual species such as Arabidopsis may be exposed to sufficiently cold and humid summer days to break dormancy and trigger germination thereby bypassing the first checkpoint. The ABA-mediated growth arrest described here would then increase the chance of the germinated seedlings surviving in the event of a subsequent prolonged drought. Accordingly, abi5-4 seeds were also able to germinate and green in 200 mM NaCl indicating that high salt concentration cannot arrest growth in these mutants. Third, ABI5 expression is regulated by drought and salt exposure. In WT plants, ABI5 abundance closely reflects environmental conditions during the 60 hours following stratification. Within this time period, ABI5 levels increase upon imposition of water stress and decrease upon stress removal. Fourth, in addition to being necessary for the ABA-mediated growth arrest, ABI5 overexpression results in an enhanced response to ABA indicating that in planta, ABI5 limits transduction of a stress-responsive signal.

The present work shows that ABI5 accumulation per se is not sufficient to arrest growth because transgenic plants overexpressing ABI5 grow normally in the absence of ABA. ABI5 could be activated by recruitment of a partner protein, by nucleocytoplasmic shuttling or by phosphorylation. In the latter case, the ABI5 mobility shift indicates that ABI5 is phosphorylated de novo upon ABA exposure. However, the number of phosphate groups per ABI5 molecule appears to be unchanged. Therefore, our data do not rule out whether the mobility shift is a result of a novel and unique phosphorylation event on newly translated ABI5 or a rearrangement of phosphate groups in a given ABI5 molecule. In the latter case, ABI5 would then be dephosphorylated and phosphorylated in novel sites in vivo.

The results presented herein teach one how to direct manipulation of seed germination and seedling hardiness. The ability to engineer the earliest and most critical events of the plant life cycle will not only have a substantial effect on agriculture (e.g., crop production or weed control), but might also be of considerable ecological significance.

The following definitions are used for the present disclosure.

A “seed, seedling or plant” of the present disclosure is one which is sensitive to abscisic acid. The phrase “seed, seedling or plant” includes all stages in the life of a plant and includes somatic embryos and primed seeds.

“Drought tolerant” means that the seeds, seedlings or plants which are drought tolerant are able to delay germination (seeds) or live longer (seedlings and plants) as compared to wild type seeds, seedlings or plants in the absence of or deprivation of water.

Drought conditions means a lack of water wherein said lack of water results in death of wild type seedlings within 2 days in the absence of added abscisic acid.

A plant is tolerant or resistant to high salt if it grows in high salt as well as or better than a wild type plant grows in media of normal salt such as the salt concentration in Murashige and Skoog medium (Murashige and Skoog, 1962).

High salt means a salt concentration equal to or greater than 200% of the salt concentration of Murashige and Skoog medium (Murashige and Skoog, 1962).

An activatable promoter is a promoter which is made active by adding a component which causes a gene under the control of said promoter to become active. This definition of an activatable promoter comprises inducible promoters and derepressible promoters.

A seed, seedling or plant which is hypersensitive to ABA is a plant which shows the same phenotype as a wild type seed, seedling or plant at a concentration of ABA less than that required for the wild type. For example, if a wild type seed, seedling or plant requires 5 μM ABA to exhibit a specified phenotype a hypersensitive plant would exhibit the same phenotype at a concentration which normally does not produce noticeable effects in wild type plants, for example at 0.5 μM. The response depends upon the amounts of ABI5 produced and the amount of ABA present. A hypersensitive plant will exhibit the same phenotype as a wild-type plant at a concentration 3-fold or less of ABA as used on the wild-type plant.

A seed, seedling or plant which overproduces ABI5 is one which produces 2-fold or greater more ABI5 as compared to a wild type seed, seedling or plant, respectively, under identical conditions as the overproducing seed, seedling or plant. If the seed, seedling or plant is one with an ABI5 gene under the control of an activatable promoter, the seed, seedling or plant is one which overproduces ABI5 (2-fold or greater as compared to wild type) in the presence of an activator of said promoter, but this seed, seedling or plant need not overproduce ABI5 in the absence of an activator.

By long term storage is meant storage of at least 6 months, preferably storage of at least one year, and more preferably storage of three or more years.

The present invention is further detailed in the following Examples, which are offered by way of illustration and are not intended to limit the invention in any manner. Standard techniques well known in the art or the techniques specifically described below are utilized.

ABA induces ABI5 expression early in development

Seeds of Arabidopsis thaliana, ecotype Wassilewskija, Columbia or Landsberg erecta were surface sterilized in 50% commercial bleach (Clorox) and 0.05% Tween 20 as described (Lopez-Molina and Chua, 2000) and then plated on Murashige and Skoog (MS) medium (Murashige and Skoog, 1962) containing 0.8% (w/v) Bacto-Agar (Difco Laboratories). Plates were routinely kept for 3 days in the dark at 4° C. to break dormancy (stratification) and transferred thereafter to a tissue culture room under constant white fluorescent light (27 μmol.m⁻².s⁻¹) at 22° C. Seeds of abi1, abi3-I and abi4 were obtained from the Nottingham Arabidopsis Stock Center (NASC). For ABA treatments, seedlings were transferred to plates supplemented with ABA (mixed isomers, Sigma A7393) which was dissolved in methanol. Control plates contained equal amounts of methanol.

abi5-4 mutants were selected on the basis of their ability to germinate and grow in otherwise inhibitory ABA concentrations (Lopez-Molina and Chua, 2000). In the absence of ABA, seed germination and plant growth were indistinguishable between WT Ws and abi5-4. We hypothesized that ABI5 could act as a repressor of germination and growth, and that ABA might trigger its accumulation. To this end, we used Northern and Western blot analyses to investigate whether ABA would regulate ABI5 transcript levels and protein expression during the first few days post-stratification.

RNA extraction and Northern blot hybridizations were done as described (Lopez-Molina and Chua, 2000). A DNA fragment encoding the ABI5 open reading frame was cloned into pET28a (Novagen) and the recombinant protein was induced and purified using a commercial kit according to the manufacturer's instructions (His trap, Amersham Pharmacia). Polyclonal anti-ABI5 antiserum was obtained from rabbits immunized with His-tagged ABI5. Standard methods using His-ABI5 coupled to a CNBr activated Sepharose 6-MB column (Amersham Pharmacia) were used to affinity purify specific ABI5 antibody from sera.

Plant protein extracts were resolved under reducing conditions using 10% SDS-polyacrylamide gels (Laemmli, 1970). Proteins were transferred onto PVDF membranes (Immobilon-P, Millipore) which were incubated with primary affinity-purified-ABI5 antibody (diluted 1:3,000, 60 μg/mL) and secondary antibodies, peroxidase-conjugated anti-rabbit (Boehringer Mannheim; diluted 1:3,000), for 1 hour at room temperature in TBS (25 mM Tris-HCl at pH 7.4; 137 mM NaCl, 5 mM KCl) supplemented with 4% non fat dry milk. After incubation, membranes were washed twice (10 minutes each) with TBS containing 0.05% Tween 20. After the final wash, membrane-associated peroxidase activity was visualized using the ECL kit (Boehringer Mannheim).

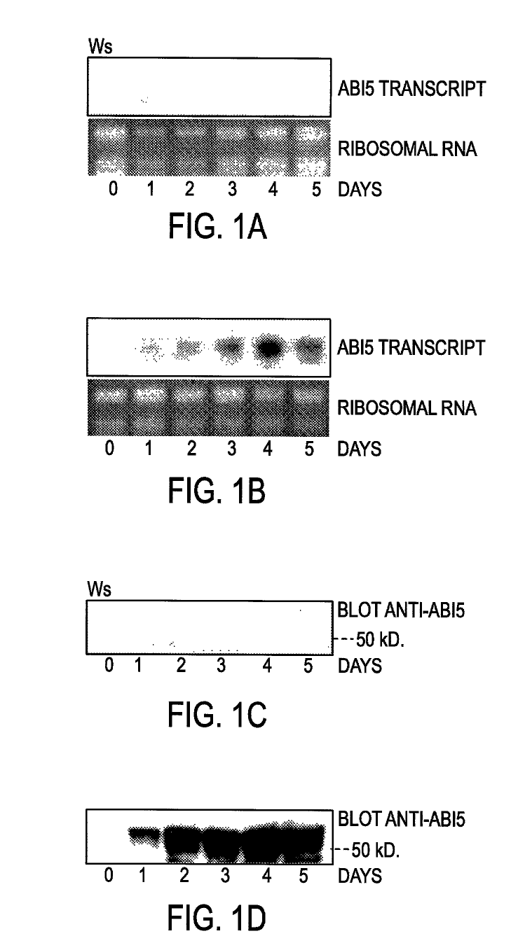

ABA concentrations (3-10 μM) that discriminate between abi5-4 mutants and WT under normal growth conditions were selected. WT Wassilewskija (Ws) seeds were plated on a germination medium in the presence or absence of 5 μM ABA and kept in the dark at 4° C. for 3 days to break dormancy (stratification) before being transferred to 22° C. under light for 1, 2, 3, 4 and 5 days. Total protein and RNA were isolated. As shown in FIG. 1A (each lane contains 3 μg total RNA), ABI5 transcript was undetectable in the absence of ABA but was induced after one day in the presence of ABA (0 indicates the time immediately following transfer). FIG. 1B shows that ABA also induced accumulation of ABI5 (each lane contains 10 μg protein). Indeed, by immunoblot analysis, the ABI5 protein with a molecular mass of about 50 Kd was detected two days after stratification in the presence of ABA. This is in agreement with the predicted Mr of 47,000 for ABI5. Control experiments with abi5-4 null mutants confirmed that this band is specific. In the absence of ABA, this protein was absent (FIG. 1B) and undetectable even after prolonged film exposure. In the presence of ABA, the protein level increased at least 10-fold during the first two days and remained constant thereafter. To rule out the possibility that the absence of the ABI5 protein might be due to a dilution caused by protein accumulation during the first 5 days without ABA, we compared total protein levels in Arabidopsis plants grown in the presence or absence of ABA for 6 days and found only a twofold difference in their protein content. As this is insufficient to explain the observed differences in ABI5 accumulation level, we conclude that ABI5 accumulation is strongly induced by ABA.

ABA stabilizes ABI5

Given the strong ABA-dependent ABI5 protein accumulation, we were interested to determine whether ABI5 protein stability is altered in the presence of ABA. For this purpose we generated WT/35S::HA-ABI5 transgenic lines constitutively expressing HA-ABI5 transcript. DNA manipulations were performed according to standard methods (Sambrook et al., 1989). 35S constructs were generated using a binary vector (Kost et al., 1998). Transgenic Arabidopsis lines (Ws ecotype) were generated using the Agrobacterium tumefaciens vacuum-infiltration method (Bechtold and Pelletier, 1998). Seeds (T1) from infiltrated plants were plated on MS medium containing 10 mg/L gluphosinate ammonium (Crescent Chemical) and 100 mg/L cefotaxime (Sigma).

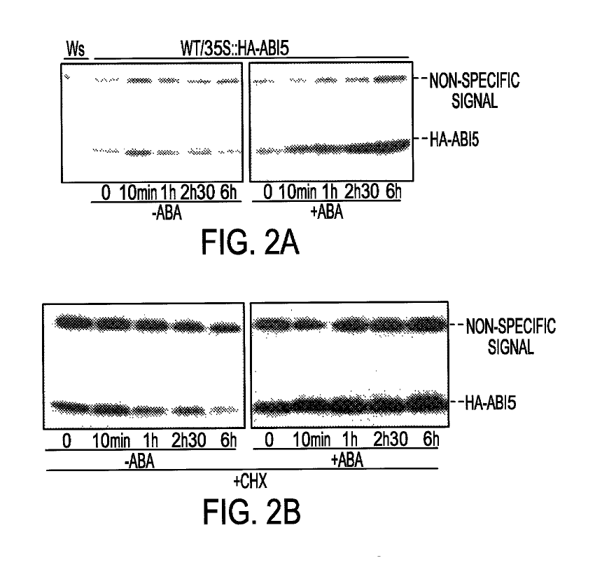

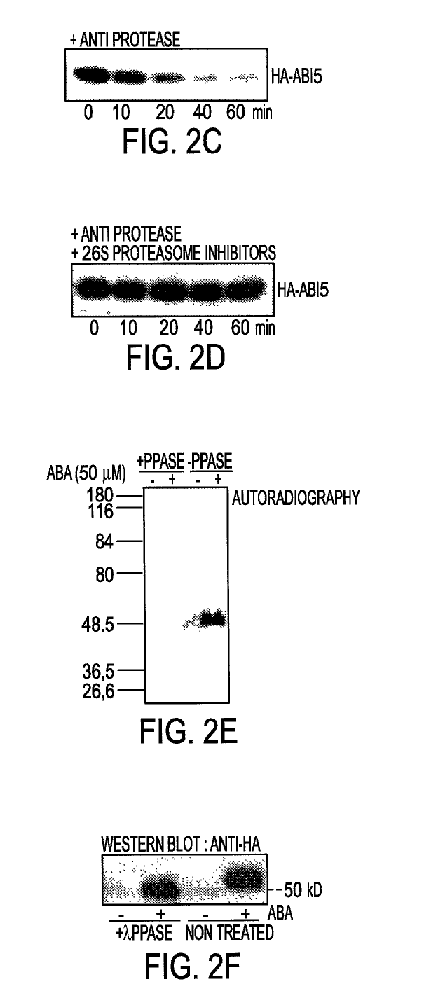

In these lines, HA-ABI5 accumulation is 5-10 fold higher in the presence of ABA as shown by immunoblot analysis using anti-HA antibodies. 8-day old Ws/35S::HA-ABI5 seedlings (T3 generation) were transferred to MS medium with or without 50 μM ABA. Proteins were extracted at 0 hours, 10 minutes, 1 hour, 2 hours and 30 minutes, and 6 hours and ABI5 protein levels were monitored by Western blot analysis using antibodies to HA (FIG. 2A; each lane contains 5 μg protein). Similar results were obtained at all stages of development in abi5-4/35S::ABI5 transformants using antibodies to ABI5. This indicates that ABA regulates ABI5 accumulation post-transcriptionally since the transgene RNA levels were unaffected by ABA. Several mechanisms can be proposed to explain this observation, including an increase in protein synthesis and/or stability. To test the protein stability hypothesis, 8-day old WT/35S::HA-ABI5 transgenic seedlings were treated with the protein synthesis inhibitor cycloheximide for 15 hours before addition of ABA. Eight-day old WT/35S::HA-ABI5 transgenic seedlings were incubated in liquid MS medium supplemented with 100 μM cycloheximide. After 15 hours, 50 μM ABA was added to the medium and proteins were extracted at the indicated times. Equal amounts (5 μg) of protein were loaded on a 10% SDS-polyacrylamide (Novex) gel and analyzed by Western blots using rabbit antibody to HA (1:3,000; Santa Cruz). FIG. 2B shows that ABA treatment prevented the decrease in ABI5 level observed in untreated plants, indicating that ABA prevents ABI5 degradation.

To better understand the mechanism underlying this phenomenon, we measured ABI5 levels using a cell-free assay (Osterlund et al., 2000). Eight-day old WT/35S::HA-ABI5 transgenic seedlings were ground in liquid nitrogen and proteins were extracted. Cell debris was pelleted by centrifugation and equal amounts of extract were transferred to individual tubes with a cocktail of anti-protease (complete Mini, Amersham) supplemented with 1 mM PMSF with or without 26S proteasome-specific inhibitors ALLN, MG115, MG132, PSI (10 μM each, Calbiochem). Samples were incubated at 30° C. and an equal volume of SDS-loading buffer was added to stop the reactions. Equal amounts of sample were then analyzed by Western blots (rabbit polyclonal antibody to HA).

We observed a rapid degradation of ABI5 within 1 hour even in the presence of a mixture of non-specific protease inhibitors (FIG. 2C). However, this degradation can be prevented by inhibitors specific to the 26S proteasome (FIG. 2D) indicating that the latter is responsible for the degradation.

ABA induces ABI5 phosphorylation

In addition to the ABI5 accumulation effect observed upon ABA exposure in WT/35S::HA-ABI5 transgenic lines, we systematically observed a slight decrease in the mobility of the HA-ABI5 protein, as compared to the protein produced in the absence of ABA exposure, on SDS-PAGE Western blots using rabbit polyclonal antibody to HA. This effect was rapid and was observed in seedlings treated with ABA at all developmental stages. Indeed, after 10 minutes of ABA treatment, the mobility shift was already apparent and persisted for at least 48 hours. To test whether this mobility shift is due to ABA-triggered phosphorylation of ABI5, ABA treated 8-day old seedlings were incubated with [32p] orthophosphoric acid. Eight-day old WT/35S::HA-ABI5 transgenic seedlings (T3 generation) were treated in liquid MS medium with [³²P] orthophosphoric acid (100 μCi, 285.5 Ci/mg, NEN) for 90 minutes. Seedlings were further treated with or without 50 μM ABA for 30 minutes.

HA-ABI5 was immunoprecipitated using rabbit antibody to HA coupled to beads according to Ausubel et al. (1999) for 5 hours at 4° C. using 5 μg of rabbit antibody to HA coupled on agarose beads (Santa Cruz). Subsequently, the immunoprecipitates were treated with recombinant lambda phosphatase. Beads were washed three times with 50 mL of immunoprecipitation buffer and once with 0.5 mL of λ phosphatase buffer (New England Biolabs). Beads were resuspended with 50 μL of λ phosphatase buffer and divided in two portions: one portion was treated with 200 U of λ phosphatase for 10 minutes at 30° C.; the other portion was not treated. An equal volume of SDS-loading buffer was added to stop the reactions. Proteins were separated by SDS-PAGE for autoradiography and Western blot analysis. Equal amounts of sample (10% of the total) were analyzed by Western blot using mouse monoclonal antibody against HA (Santa Cruz) as first antibody. The remaining sample was analyzed by SDS-PAGE and Phosphorlmager (BioRad) was used to determine relative radioactive incorporation.

FIG. 2D shows that ABI5 is phosphorylated in vivo both in the presence and absence of ABA, although the specific activity of ABI5 does not increase upon ABA exposure (FIG. 2E). The phosphatase treatment was sufficient to remove the radiolabeling (FIG. 2E) and to restore the mobility of the HA-ABI5 protein to that of HA-ABI5 in protein extracts of control plants not treated with ABA (FIG. 2F).

The ABA-dependent ABI5 accumulation occurs within a limited developmental time window post-stratification

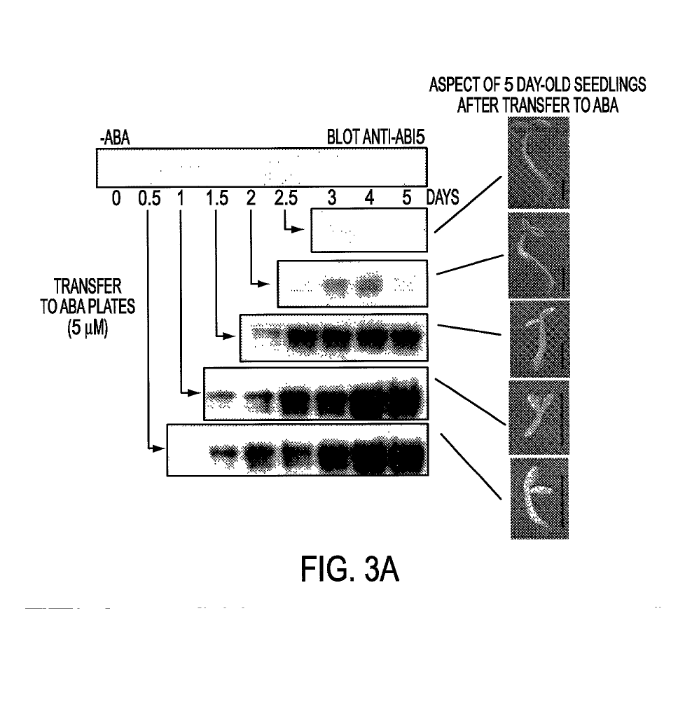

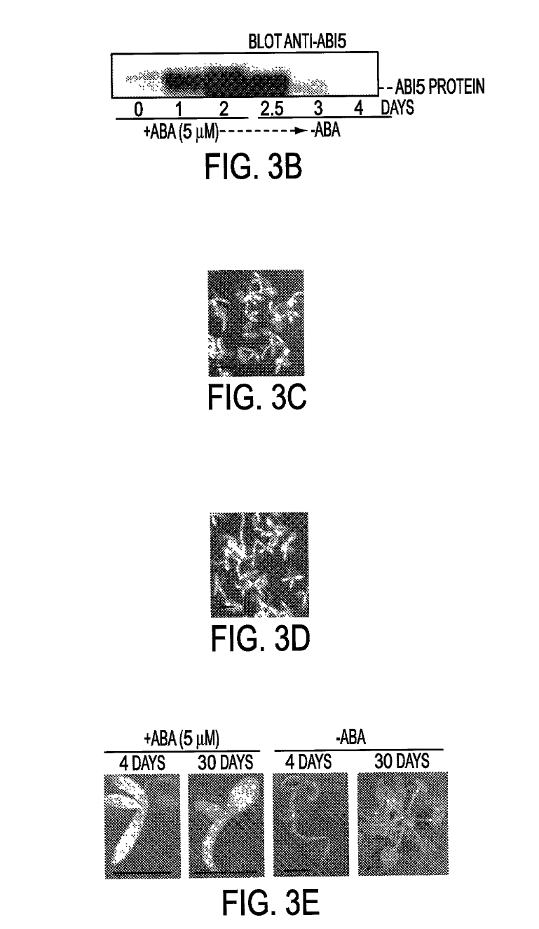

To determine whether the observed ABA-dependent accumulation of ABI5 levels was restricted to early development, we stratified WT seeds in the absence of ABA and then transferred the seedlings at different time intervals to a medium supplemented with 5 μM ABA (FIG. 3A). WT Ws seeds were kept in darkness at 4° C. for 3 days without ABA and then transferred to 5 μM ABA plates at the indicated times post-stratification. ABI5 levels were monitored by Western blot analysis. Photographs depict representative seedlings 5 days after the different treatments. FIG. 3A shows that ABI5 accumulation was efficiently induced during a limited developmental window between 48-60 hours post-stratification. After 60 hours, little or no induction was observed. FIG. 3B shows that ABA removal was accompanied by a rapid decrease in ABI5 levels. WT Ws seeds treated with ABA (5 μM) as described for the experiments shown by FIG. 1B were transferred to plates without ABA 2 days post-stratification. ABI5 levels were monitored by Western blot analyses as described for FIG. 1 (FIG. 3B).

ABA Inhibition of Root Growth and Water Loss

The following methods were used in obtaining the further results described below. WT Ws seedlings and transgenic Ws seedlings carrying 35S::HA -ABI5 (WT/35S::HA -ABI5, T3 generation) were germinated and grown for 5 days on MS medium without ABA. Seedlings were then transferred to plates containing 5 μM ABA, and subsequent root growth was scored after 5 days. Values were expressed as the average ±SD of 15 seedlings. For water loss measurement, young rosette leaves at the same developmental stage were excised from WT Ws, WT/35S::HA-ABI5 and abi1 plants and weighed at the different times indicated (n=3).

ABA efficiently triggers growth arrest within a limited developmental time window post-stratification

Given the ABA-insensitivity of abi5-4 mutant and the timing of ABI5 induction upon ABA exposure, we studied the effect of ABA on growth during early development. Application of 5 μM ABA within 60 hours post-stratification did not prevent germination but maintained the germinated embryos in an arrested state for several days, whereas ABA applied outside the 60-hour time frame failed to arrest growth and prevent greening (FIG. 3A). Virtually all WT seeds were fully germinated on 5 μM ABA only 5 days post-stratification (FIG. 3C). Similar results were obtained with different Ws seed batches and for the ecotypes Landsberg and Columbia. At 10 μM ABA, complete germination was observed 8 days post-stratification (FIG. 3D). Thereafter, ABA efficiently blocked further growth for at least a month (FIG. 3E). ABI5 levels remained constant as long as ABA was present. During this period, almost all embryos (95%±3, n=200) remained white and quiescent, and did not grow. Upon ABA removal, all of the embryos started to green and resumed normal growth.

Taken together, these results indicate that ABA is more efficient as an early growth inhibitor than as an inhibitor of germination. They also show that ABA, in addition to delaying germination, can reversibly block growth during a narrow developmental time interval following germination and before the onset of vegetative growth.

ABI5 expression is induced by drought and high salt stress

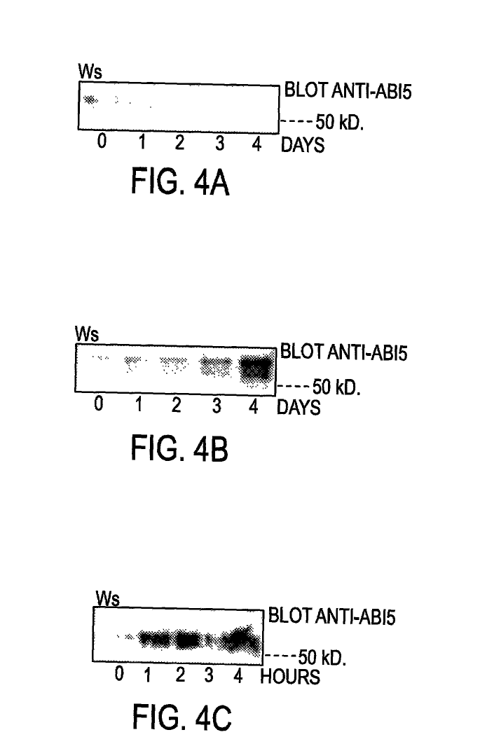

Since ABA mediates stress responses in Arabidopsis we examined ABI5 expression upon water stress. ABI5 accumulation was measured in seeds treated with high salt concentrations or subjected to water deprivation. Both treatments resulted in a rapid and robust increase of ABI5 accumulation (FIGS. 4A-C). WT Ws seeds were kept in darkness at 4° C. for 3 days with or without NaCl (200 mM) and then transferred to constant light at 22° C. for 1, 2, 3, 4 and 5 days (0 indicates the time immediately following transfer). Western blot analyses using antibodies to ABI5 were performed as described for the results shown in FIG. 1B. FIG. 4A shows results in the absence of added NaCl. FIG. 4B shows the results in the presence of 200 mM NaCl. FIG. 4C shows the results of plants subjected to water deprivation. One day-old seeds were transferred to absorbing paper for 1, 2, 3 or 4 hours and Western blot analyses using antibodies to ABI5 were performed as described for the experiments shown in FIG. 1 B.

Germinated and ABA-arrested mature embryos are resistant to extended drought

The induction of ABI5 accumulation by drought indicated that this protein plays a protective role under this condition. To test this hypothesis, we broke embryo dormancy of WT and abi5-4 seeds in the absence of ABA for 24 hours. Seeds were then transferred for an additional 10 days to media with or without ABA. After 10 days, ABA-treated WT mature embryos had germinated but were arrested in growth as described above, whereas abi5-4 or non-treated WT Ws plants were green and continued to grow. Plants were then water-stressed for different time intervals on absorbent papers before being allowed to recover in normal medium. Table 1 shows that a majority of the quiescent WT mature embryos were able to survive after 36 hours of water stress. This is in sharp contrast with abi5-4 embryos, none of which survived. abi5-4 plants were able to survive better with than without ABA (Table 1), although not to the same extent as WT. This observation probably reflects the fact that abi5-4 (like all other abi mutants) is not completely insensitive to ABA, indicating that ABI5 is not the only protein mediating ABA-dependent processes early in development. Indeed, abi5-4 plants grow more slowly on 5 μM ABA than on medium lacking it, whereas the growth of WT plants is completely arrested. This developmental difference may be responsible for the higher resistance to water depletion of ABA-treated abi5-4 plants (Table 1).

TABLE 1
Survival Rate of WT Ws and abi5-4 Plants Treated with or without ABA
for 10 Days and Transferred to Absorbing Papers for the Times
Indicated (n = 30)

| | | Percentage of survival after different | | | | |
|---|---|---|---|---|---|---|
| | | period of drought treatment (hours) | | | | |
| | | 0 | 8 | 12 | 24 | 36 |
| Ws | −ABA | 100 | 53 | 2 | 0 | 0 |
| | +ABA(5 μM) | 100 | 100 | 98 | 90 | 84 |
| abi5 | −ABA | 100 | 50 | 0 | 0 | 0 |
| | +ABA(5 μM) | 100 | 97 | 55 | 20 | 0 |

ABI5 overexpressing plants are hypersensitive to ABA

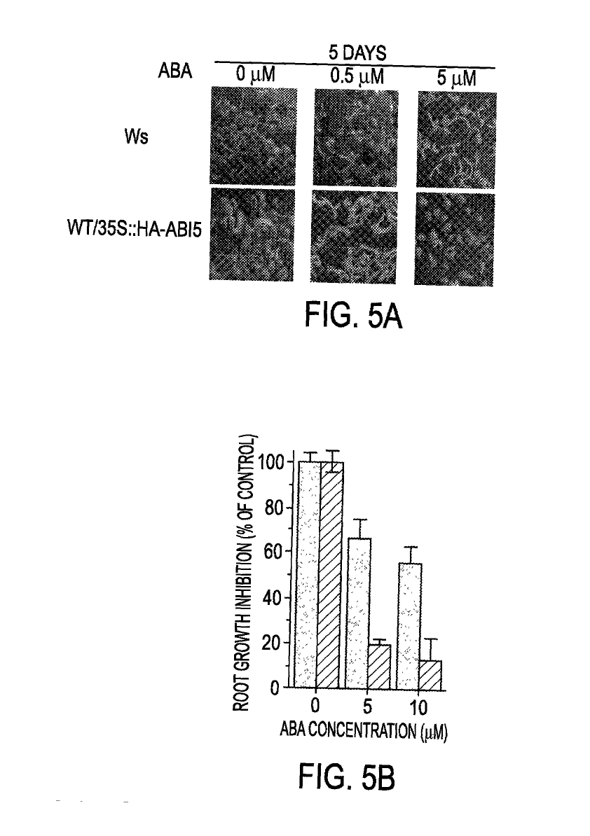

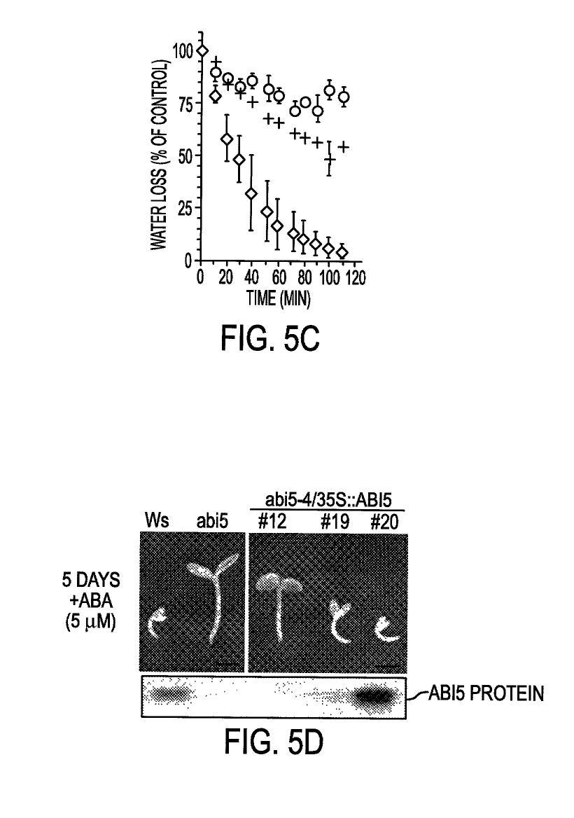

To determine whether the presence of ABI5 is sufficient to arrest growth in the absence of ABA, we expressed the ABI5 gene from a constitutive promoter. Transgenic Arabidopsis lines expressing ABI5 under the control of the cauliflower mosaic virus 35S promoter in WT Ws or abi5-4 backgrounds were generated and several independent lines expressing various levels of ABI5 were obtained. WT Ws and Ws/35S::HA-ABI5 transgenic plants were grown for 5 days with 0, 0.5 and 5 μM ABA. In the transgenic line shown in FIG. 5A, ABI5 levels in absence of ABA were at least five fold higher than that in WT plants grown in the presence of ABA. Transgenic seeds were arrested in development at 0.5 μM ABA and only started to germinate after 5 days (FIG. 5A). WT Ws seeds were insensitive to 0.5 μM ABA and were fully germinated in 5 μM ABA after 5 days (FIG. 5A). Despite the accumulation of ABI5, the lines germinated and developed normally in the absence of ABA. These results show that the presence of ABI5 is necessary, but not sufficient for growth arrest and that ABA is required for a further activation step.

We next investigated the effect of ABA in ABI5 overexpressing lines. Addition of 5 μM ABA severely delayed germination by at least 7 days and blocked mature embryo growth in several lines. To measure more precisely the ABA response, we plated WT/35S::HA-ABI5 transgenic seeds on growth media containing different ABA concentrations. Treatment of WT/35S::HA-ABI5 plants with 0.5 μM ABA was sufficient to phenocopy the response of WT plants exposed to 5 μM ABA (FIG. 5A).

At 0.5 μM ABA, WT plants grew normally (FIG. 5A). Inhibition of root growth by ABA was unaffected in abi5-4 mutants but was exacerbated in ABI5-overexpressing lines (FIG. 5B). Five-day old seedlings grown in the absence of ABA were transferred to 0, 5 and 10 μM ABA plates for 5 days. The root length of ABA-treated seedlings was expressed as a percentage of non-treated controls (n=15). FIG. 5B shows that inhibition of root growth by exogenous ABA is exacerbated in Ws/35S::HA-ABI5 transgenic lines (light bars, right side of each pair) as compared to WT Ws plants (dark bars, left side of each pair).

ABI5-overexpressing plants also retained water more efficiently than WT plants (FIG. 5C). Ws/35S::HA-ABI5 (open circles) transgenic plants retained water more efficiently. Young rosette leaves from WT Ws control (crosses) and transgenic plants at the same developmental stge (one month) were excised and weighed at different times as indicated in FIG. 5C (n=3). abi1 measurements (lozenges) are included as a control for water loss. These results show that transgenic plants overexpressing ABI5 are hypersensitive to ABA.

The extent of growth arrest is correlated with ABI5 protein levels

We used abi5-4 lines expressing the 35S::ABI5 transgene (abi5-4/35S::ABI5) to investigate the relationship between protein accumulation and the extent of growth arrest in response to ABA. Seeds from WT Ws, abi5-4 and three independent abi5-4 transgenic lines carrying a 35S::ABI5 transgene were treated with 5 μM ABA as described for the experiments shown in FIG. 1B. FIG. 5D depicts representative seedlings after 5 days. Western blots (lower portion of FIG. 5D) using anti-ABI5 antibodies were performed on these same plants. FIG. 5D shows that abi5-4/35S::ABI5 transgenic lines expressing intermediate ABI5 levels also displayed an intermediate phenotype in the presence of ABA. These results indicate that ABI5 is a rate-limiting factor conveying the ABA-mediated developmental growth arrest.

The results of the above experiments show that overexpression of ABI5 in seeds, seedlings and plants confers resistance or tolerance to both drought and high salt. In addition, overexpression of ABI5 results in seeds, seedlings and plants which are hypersensitive to ABA and which respond to low levels of ABA which levels have no or only minor effect upon wild type plants. Overexpression of ABI5 will therefore protect seeds, seedlings or plants against stress such as drought or high salt.

A variety of approaches to take advantage of the relationship between ABI5 production, ABA and stress are available. Overexpression of ABI5 may be desired to delay germination or to arrest the growth of germinated seeds. This desire for delay can occur if weather conditions would cause plants to germinate and grow at an inopportune time, such as too early or prior to the normal growing season because of unusual weather. Plants which overexpress ABI5 could be inhibited in their germination and growth by an application of ABA. Because overexpression of ABI5 makes seeds, seedlings and plants hypersensitive to ABA, lower levels of ABA would be required as compared to ABA application to wild type plants. In addition, ABI5 expression can be either under the control of a constitutive promoter or it can be under the control of an activatable promoter, e.g., a promoter which can be induced or derepressed. Transgenic seeds, seedlings and plants which use a constitutive promoter for ABI5 expression can be manipulated by adjusting the amount of ABA to which they are exposed. Transgenic seeds, seedlings and plants which use an activatable promoter can be manipulated by activating the promoter and/or adjusting the amount of ABA to which they are exposed. By activating the promoter to overexpress ABI5 it becomes possible to utilize lower levels of ABA as compared to levels required to obtain the same phenocopy in a wild type plant. Activatable promoters for plants are well known by those of skill in the art. See, for example, Zuo and Chua (2000), Zuo et al. (2000), Aoyama and Chua (1997), which are incorporated herein by reference. Using activatable promoters can be advantageous for certain purposes, e.g., it allows one to increase ABI5 production during times of stress such as drought but not during other times, e.g., with a constitutive promoter plant growth may be inhibited at times when it would be preferred for the plant to be growing normally. Of course, it is the combination of overexpression of ABI5 in conjunction with the levels of ABA which effect the response to stress, overexpression of ABI5 alone not being sufficient.

Other uses of this technology can easily be envisioned by those in the agricultural industry. For example, elite clones of tree species are propagated vegetatively either by organogenesis or somatic embryogenesis. In the case of conifers, vegetative cutting does not provide enough multiplication factor within the time constraint. Therefore somatic embryogenesis is the preferred method of propagation. Several companies sell somatic embryos (SEs) to forestry companies for large scale planting of elite clones. An important step in making SEs is dehydration of the SEs for storage and subsequent germination. This step is done with ABA treatment. Overexpression of ABI5 will reduce the amount of ABA required because of hypersensitivity of the embryos to ABA. In addition, overexpressing germinated seedlings will be better protected if they encounter unfavorable conditions such as water stress.

When somatic embryos go through a maturation process in vitro the process involves gradual dehydration and addition of ABA, simulating the physiological conditions of zygotic embryos during seed maturation. During natural zygotic embryo formation, ABI5 is synthesized late during embryogenesis and maintains dormancy in the embryo during storage and before germination. If the somatic embryos are not matured properly, they will not survive long term storage and will not germinate into healthy seedlings. ABI5 can serve as a molecular marker for somatic embryos that are matured properly. Somatic embryos that do not accumulate ABI5 will not respond properly to ABA and dehydration in vitro and will likely not survive long term storage.

In many species, particularly in vegetable crops, the seeds are primed before being sold to farmers in order to ensure a high germination frequency. This means that the seeds are allowed to imbibe water up to a certain stage and then they are dehydrated again to make them quiescent. Such seeds which overexpress ABI5 will be better able to survive during storage.

While the invention has been disclosed in this patent application by reference to the details of preferred embodiments of the invention, it is to be understood that the disclosure is intended in an illustrative rather than in a limiting sense, as it is contemplated that modifications will readily occur to those skilled in the art, within the spirit of the invention and the scope of the appended claims.

### List of References

Aoyama T and Chua N-H (1997). The Plant J. 11:605-612.

Ausubel F M et al. (1999). Short Protocols in Molecular Biology (Wiley, New York).

Bechtold N and Pelletier G (1998). Methods Mol. Biol. 82:259-266.

Choi H et al. (2000). J. Biol. Chem. 275:1723-1730.

Finkelstein R R (1994). Plant J 5:765-771.

Finkelstein R R et al. (1998). Plant Cell 10:1043-1054.

Finkelstein R R and Lynch T J (2000). Plant Cell 12:599-609.

Giraudat J et al. (1992). Plant Cell 4:1251-1261.

Gosti F et al. (1999). Plant Cell 11:1897-1910.

Himmelbach A et al. (1998). Philos. Trans. R. Soc. Lond. B Biol. Sci. 353:1439-1444.

Kim S Y et al. (1997). Plant J 11:1237-1251.

Kornneef M et al. (1984). Physiol Plant 61:377-383.

Kost B et al. (1998). Plant J 16:393 -401.

Laemmli U K (1970). Nature 227:680-685.

Leung J et al. (1994). Science 264:1448-1452.

Leung J et al. (1997). Plant Cell 9:759-771.

Lopez-Molina L and Chua N H (2000). Plant Cell Physiol. 41:541-547.

Meyer K et al. (1994). Science 264:1452-1455.

Murashige T and Skoog F (1962). Physiol. Plant. 15:473-497.

Osterlund M T et al. (2000). Nature 405:462-466.

Parcy F et al. (1994). Plant Cell 6:1567-1582.

Parcy F and Giraudat J (1997). Plant J 11:693-702.

Sambrook J et al. (1989). Molecular Cloning: a Laboratory Manual (Cold Spring Harbor Laboratory Press, Cold Spring Harbor, N.Y.).

Uno Y et al. (2000). Proc. Natl. Acad. Sci. USA 97:11632-11637.

Wang H et al. (2000). Plant J 24:613-623.

Zuo J and Chua N-H (2000). Curr. Opin. Biotechnol. 11:146-151.

Zuo Jet al. (2000). Plant J 24:265-273.

What is claimed is:

1. A method of delaying germination of a seed, said method comprising expressing ABI5 in said seed prior to germination such that said seed overproduces ABI5 as compared to wild type seeds.

2. The method of claim 1 wherein said seed constitutively overexpresses ABI5.

3. The method of claim 1 wherein said seed comprises an ABI5 gene under the control of an activatable promoter.

4. A method of inhibiting growth of germinated embryos, said method comprising overexpressing ABI5 in said germinated embryo such that said seed overproduces ABI5 as compared to wild type germinated embryos.

5. The method of claim 4 wherein said germinated embryo constitutively overexpresses ABI5.

6. The method of claim 4 wherein said germinated embryo comprises an ABI5 gene under the control of an activatable promoter.

7. A method of protecting a seed, seedling or plant from drought comprising expressing ABI5 in said seed, seedling or plant such that ABI5 is overproduced as compared to a wild type seed, seedling or plant, respectively.

8. The method of claim 7 wherein said seed, seedling or plant constitutively overexpresses ABI5.

9. The method of claim 7 wherein said seed, seedling or plant comprises an ABI5 gene under the control of an activatable promoter.

10. A method of protecting a seed, seedling or plant from high salt concentration comprising overexpressing ABI5 in said seed, seedling or plant such that ABI5 is overproduced as compared to a wild type seed, seedling or plant, respectively.

11. The method of claim 10 wherein said seed, seedling or plant constitutively overexpresses ABI5.

12. The method of claim 10 wherein said seed, seedling or plant comprises an ABI5 gene under the control of an activatable promoter.

13. A method of making a seed, seedling or plant hypersensitive to abscisic acid comprising overexpressing ABI5 in said seed, seedling or plant such that ABI5 is overproduced as compared to a wild type seed, seedling or plant, respectively.

14. The method of claim 13 wherein said seed, seedling or plant constitutively overexpresses ABI5.

15. The method of claim 13 wherein said seed, seedling or plant comprises an ABI5 gene under the control of an activatable promoter.

16. A method of creating a seed, seedling or plant which is tolerant to drought wherein said method comprises manipulating the genome of said seed, seedling or plant or an ancestor of said seed, seedling or plant such that said seed, seedling or plant can produce more ABI5 during drought than will a wild type seed, seedling or plant, respectively.

17. The method of claim 16 wherein said seed, seedling or plant is made transgenic for ABI5 under the control of a constitutive promoter.

18. The method of claim 16 wherein said seed, seedling or plant is made transgenic for ABI5 under the control of an activatable promoter.

19. A method of creating a seed, seedling or plant which is tolerant to above normal salt concentrations wherein said method comprises manipulating the genome of said seed, seedling or plant or an ancestor of said seed, seedling or plant such that said seed, seedling or plant can produce more ABI5 in response to above normal salt conditions than will a wild type seed, seedling or plant, respectively.

20. The method of claim 19 wherein said seed, seedling or plant is made transgenic for ABI5 under the control of a constitutive promoter.

21. The method of claim 19 wherein said seed, seedling or plant is made transgenic for ABI5 under the control of an activatable promoter.

22. A method of creating a seed, seedling or plant which is hypersensitive to abscisic acid wherein said method comprises manipulating the genome of said seed, seedling or plant or an ancestor of said seed, seedling or plant such that said seed, seedling or plant produces more ABI5 than does a wild type seed, seedling or plant, respectively.

23. The method of claim 22 wherein said seed, seedling or plant is made transgenic for ABI5 wherein ABI5 is under the control of a constitutive promoter.

24. The method of claim 22 wherein said seed, seedling or plant is made transgenic for ABI5 under the control of an activatable promoter.

25. A seed, seedling or plant which is transgenic for ABI5.

26. The seed, seedling or plant of claim 25 wherein said seed, seedling or plant comprises an ABI5 gene under the control of a constitutive promoter.

27. The seed, seedling or plant of claim 25 wherein said seed, seedling or plant comprises an ABI5 gene under the control of an activatable promoter.

28. The seed, seedling or plant of claim 25 wherein said seed, seedling or plant overproduces ABI5.

29. The seed, seedling or plant of claim 25 wherein said seed, seedling or plant is hypersensitive to abscisic acid.

30. The seed, seedling or plant of claim 25 wherein said seed, seedling or plant is resistant to drought.

31. The seed, seedling or plant of claim 25 wherein said seed, seedling or plant is resistant to high salt.

32. A method of protecting somatic embryos from prematurely germinating comprising overexpressing ABI5 in said somatic embryos.

33. The method of claim 32 wherein said somatic embryos are treated with abscisic acid.

34. A method of protecting primed seeds from prematurely germinating comprising overexpressing ABI5 in said primed seeds.

35. The method of claim 34 wherein said primed seeds are treated with abscisic acid.

36. A method of determining whether a somatic embryo has been properly matured or primed such that it will survive long term storage, comprising measuring the amount of ABI5 present in the embro, wherein if the amount is equal to or greater than the amount of ABI5 in a zygotic embryo the somatic embryo has been properly matured.
